(19) United States
(12) Patent Application Publication
(10) Pub. No.: US 2013/0046191 A1
(43) Pub. Date: Feb. 21, 2013

(54) SYSTEM AND METHOD TO MEASURE ARTERIAL PULSE PRESSURE SIGNALS

(71) Applicants: Kang Ping Lin, Taoyuan County (TW); Hen Hong Chang, Taoyuan County (TW); Wen Chen Lin, Miaoli County (TW); Geng Hong Lin, Pingtung County (TW)

(72) Inventors: Kang Ping Lin, Taoyuan County (TW); Hen Hong Chang, Taoyuan County (TW); Wen Chen Lin, Miaoli County (TW); Geng Hong Lin, Pingtung County (TW)

(21) Appl. No.: 13/306,882

(22) Filed: Nov. 29, 2011

(30) Foreign Application Priority Data
Aug. 19, 2011 (TW) 100129817

(51) Int. Cl. A61B 5/02

(52) U.S. Cl. 600/500

(57) ABSTRACT

The present invention discloses a system and method to measure the pulse pressure signals, which inflates and deflates at least one air bag to detect pulse pressure signals of a human limb. The system comprises a detection device and a host device. The detection device is place on an artery of a human limb and includes one or more air bags. The host device controls inflation and deflation of the air bags, measures pressure variation of the air bags, and records and analyzes pulse pressure signals. The system and method of the present invention can simulate the three-finger technique of pulse diagnostics of the traditional Chinese medicine and provide physicians with more reliable pulse-diagnostics information.

### BACKGROUND OF THE INVENTION

1. Field of the Invention

The present invention relates to a system and method to measure arterial pulse pressure signals, particularly to a system and method to measure the arterial pulse pressure signals of human limbs with an array of air bags. The pulse pressure signal is referred to the pressure signal detected by a sensor from air bags in the present invention. The arterial pulse pressure signal is also called the pulse signal thereinafter.

2. Description of the Related Art

In modern medicine, there are electrocardiography, angiocardiography, ultrasonography, invasive blood pressure waveform measuring technology, etc. to diagnose cardiovascular diseases. In traditional Chinese medicine, there is also a sophisticated pulse diagnostics, which has been a subject many researches intend to scientize.

There are two arteries in a human wrist—the radial artery and the ulnar artery. The pulsation of the radial artery is easy to touch at a region below the thumb. The pulse diagnostics of the traditional Chinese medicine is normally applied to the region. The physicians of the traditional Chinese medicine examine the pulsation of the radial artery in pulse diagnostics to learn the physiological status of a human body. Some researches mention that the information of the pulsation of the radial artery correlates with the flowability and distribution of blood and the frequency and rhythm of heartbeat. Therefore, the pulse diagnostics of the traditional Chinese medicine is abundant in contents and deserves studying.

The method of the pulse diagnostics of the traditional Chinese medicine is similar to that of the blood pressure measurement of the modern medicine. Both apply external pressure to an artery and then gradually decrease the pressure to observe the reaction of the artery. In pulse diagnostics, the physician places his finger pulps on the radial artery and uses a special three-forger pressing technique to sense the variation of the pulse spectrum. The information obtained in pulse diagnostics is used to assist in diagnosing the patient. There have been apparatuses for pulse diagnostics at present. However, most of them are single-point sensing devices vertically stood on the radial artery to perform pulse diagnostics. The conventional pulse-diagnostics apparatuses cannot simulate the three-finger pressing technique precisely. Further, it is hard and laborious for the user to use the conventional pulse-diagnostics apparatuses to measure pulse spectra.

### SUMMARY OF THE INVENTION

The primary objective of the present invention is to provide a system and method to measure pulse pressure signals, wherein an array of air bags are respectively inflated or deflated to different extents to compress arteries and restrict blood flow before pulse pressure signals are measured.

Another objective of the present invention is to provide a system and method to measure pulse pressure signals, which controls the inflation and deflation of air bags to provide various compression modes to simulate the complicated finger technique of the pulse diagnostics of the traditional Chinese medicine.

A further objective of the present invention is to provide a system and method to measure pulse pressure signals, which is applied to monitoring cardiovascular physiology or scientizing the pulse diagnostics of the traditional Chinese medicine, wherein the pulse pressure signals and the related parameters are detected by a non-intrusive electronic device and used to evaluate the cardiovascular function or construct a database of pulse spectra of the pulse diagnostics, and wherein the information obtained thereby can assist in monitoring and analyzing cardiovascular physiology.

To achieve the abovementioned objectives, the present invention proposes a system and method to measure pulse pressure signals, which can quantify and record the pressure applied by a physician in pulse diagnostics, and which can simulate the empirical three-finger technique of the pulse diagnostics of the traditional Chinese medicine.

The present invention discloses a system to measure pulse pressure signals, which comprises a detection device and a host device. The detection device includes at least one air bag, which is to be placed on a human limb. The host device controls the pressure of the air bag and detects the variation of the pressure of the air bag to obtain the pulse pressure signals of the human limb.

The present invention also discloses a method to measure pulse pressure signals, which comprises steps: placing a detection device having at least one air bag on the artery of a human limb; controlling the pressure of the air bag to have a specified value; and recording the pulse pressure signals when the pressure of the air bag reaches the specified value.

### BRIEF DESCRIPTION OF THE DRAWINGS

FIG. 1 schematically shows a system to measure pulse pressure signals according to one embodiment of the present invention;

FIG. 2A schematically shows a detection device of a system to measure pulse pressure signals according to one embodiment of the present invention;

FIG. 2B schematically shows an air bag before inflation according to one embodiment of the present invention;

FIG. 2C schematically shows an air bag after inflation according to one embodiment of the present invention;

FIG. 3 is an exploded view schematically showing the structure of an air bag according to one embodiment of the present invention;

FIG. 4A is a block diagram schematically showing the architecture of a system to measure pulse pressure signals according to one embodiment of the present invention;

FIG. 4B is a block diagram schematically showing the architecture of a system to measure pulse pressure signals according to another embodiment of the present invention;

FIG. 4C is a block diagram schematically showing the architecture of a system to measure pulse pressure signals according to yet another embodiment of the present invention;

FIG. 5 schematically shows the operation of a system to measure pulse pressure signals according to one embodiment of the present invention;

FIG. 6 is a flowchart of a method to measure pulse pressure signals according to one embodiment of the present invention;

FIG. 7 schematically shows compression modes of the air bag array in FIG. 4A according to one embodiment of the present invention; and

FIG. 8 is a detailed flowchart of a method to measure pulse pressure signals according to one embodiment of the present invention.

### DETAILED DESCRIPTION OF THE INVENTION

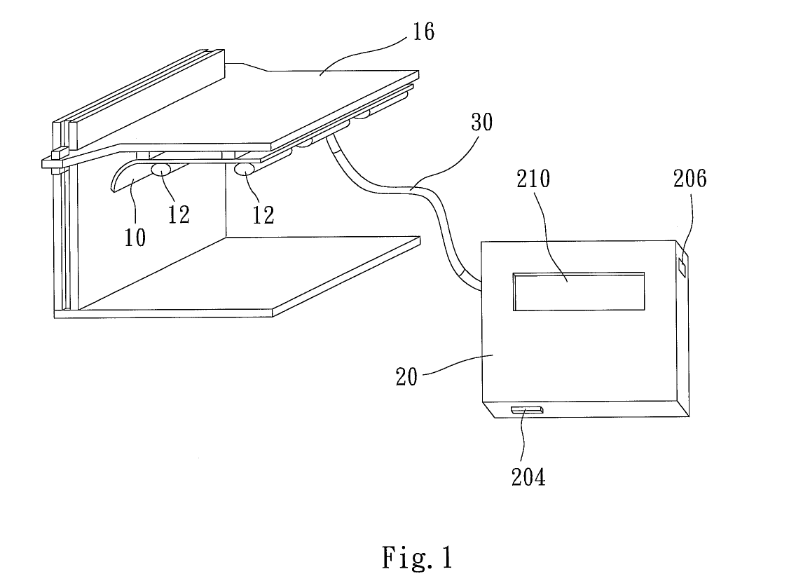

Refer to FIG. 1, which is a diagram schematically showing a system to measure pulse pressure signals according to one embodiment of the present invention. The system of the present invention is used to measure the pulse pressure signals of human limbs. Below, the present invention is exemplified by the embodiments of measuring the pulse pressure signals of human wrists. However, the present invention is not limited thereto, but can also be applied to measure the pulse pressure signals of human limbs, besides human wrists. The measurement system of the present invention comprises a detection device 10 and a host device 20. The detection device 10 includes one or more air bags 12 and connects with the host device 20 via a hose 30. In one embodiment, the host device 20 controls the pressure of the air bags 12 (including inflating and deflating air bags 12) and measures the variation of the pressure of the air bags 12, whereby the pulse pressure signals can be recorded and analyzed.

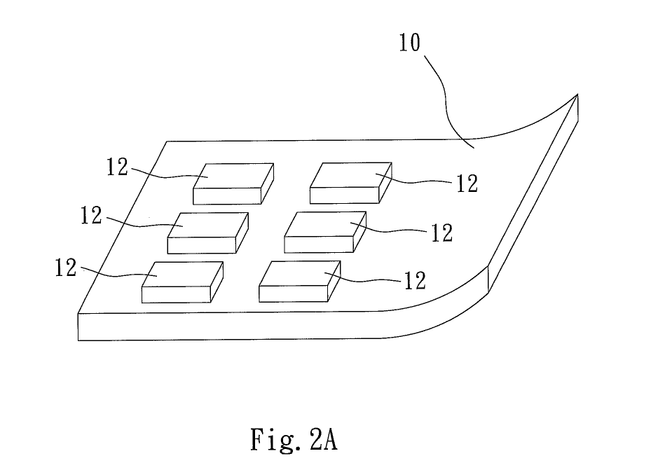

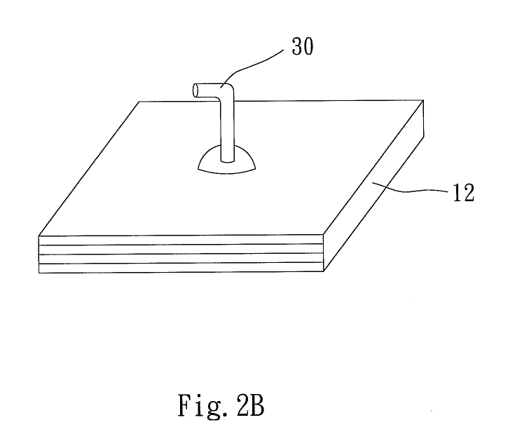

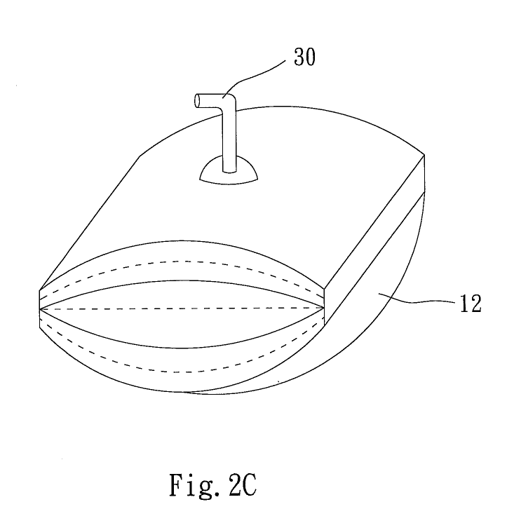

Refer to FIGS. 2A-2C. As shown in FIG. 2A, the detection device 10 has six air bags 12 arranged into a 2×3 array. However, the present invention does not restrict the detection device 10 to resemble that shown in FIG. 2A. The persons skilled in the art should be able to determine the number and arrangement of the air bags 12 according to requirement. FIG. 2B and FIG. 2C respectively show the air bag 12 which is respectively before and after inflation.

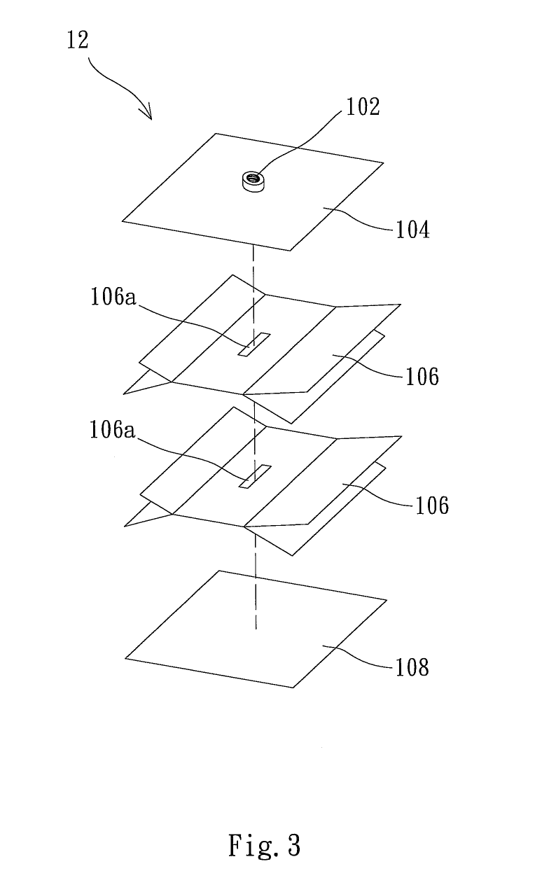

Refer to FIG. 3 for the detailed structure of the air bag. The air bag 12 includes an inlet 102, a top cover 104, a plurality of folding laminate members 106 and a bottom cover 108. The inlet 102 is connected to the hose 30. Therefore, the host device 20 can inflate or deflate the air bag 12 via the hose 30 and the inlet 102. The folding laminate members 106 are arranged between the top cover 104 and the bottom cover 108. Each folding laminate member 106 has a through-hole 106a interconnecting with the inlet 102. In one embodiment, the folding laminate member 106 is formed via assembling a plurality of lower-elasticity plastic laminates together. When the host device 20 inflates or deflates the air bag 12 to adjust the pressure of the air bag 12, the folding laminate members 106 can maintain the original shape of the air bag 12, strengthen the structure of the post-inflation air bag 12, and increase the volume of the inflated air bag 12.

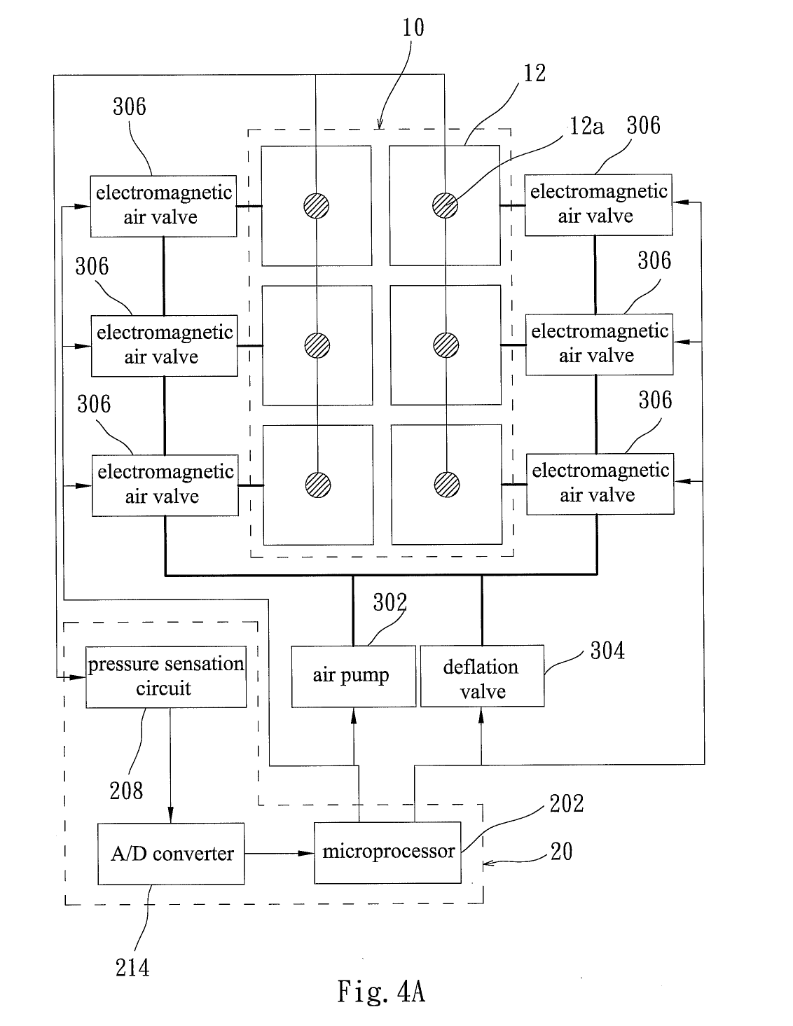

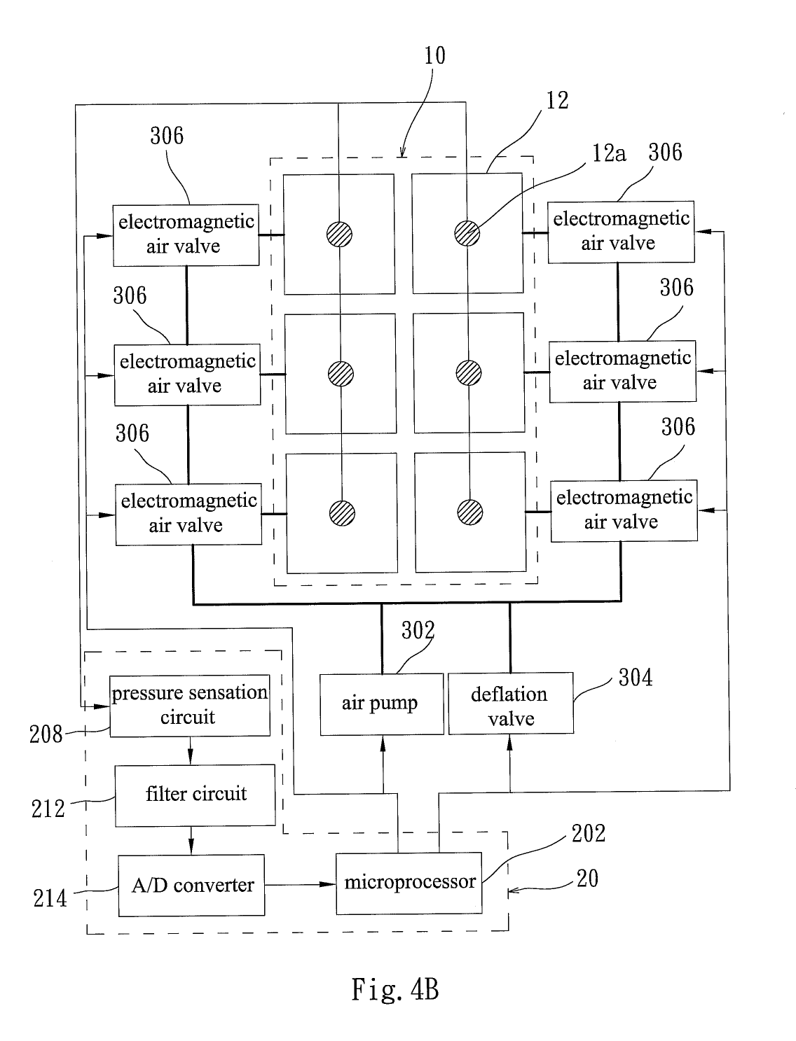

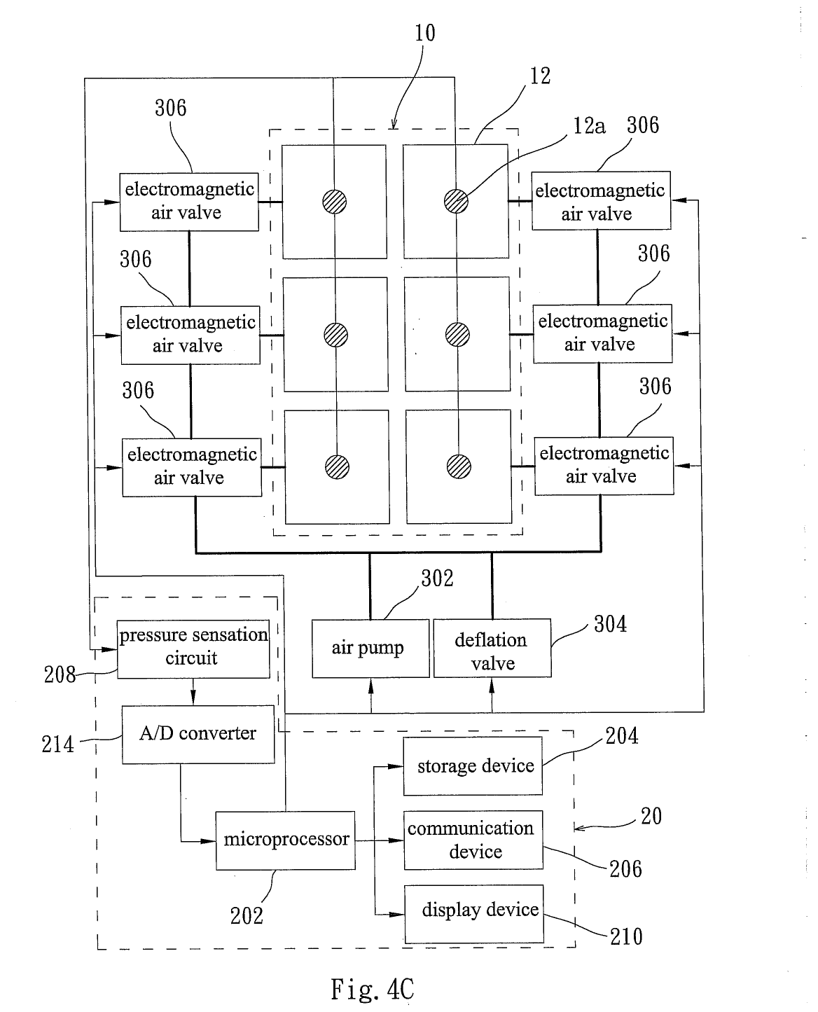

Refer to FIG. 4A, which is a block diagram schematically showing the architecture of a system to measure pulse pressure signals according to one embodiment of the present invention. The detection device 10 having 2×3 pieces of air bags 12 is used as an exemplification herein. Each air bag 12 has a pressure sensor 12a to detect the pressure of the air bag 12. The pressure sensor 12a may be but is not limited to a resistive pressure sensor. Each air bag 12 is connected with an electromagnetic air valve 306. An air pump 302 and a deflation valve 304 are further arranged between the electromagnetic air valves 306 and the host device 20. Thereby, a microprocessor 202 in the host device 20 controls the air pump 302, the deflation valve 304 and the electromagnetic air valves 306 to adjust the amount of air inflated into or deflated from each air bag 12. In the drawings, the thick line denotes the air piping, and the fine line denotes the electrical signal wire.

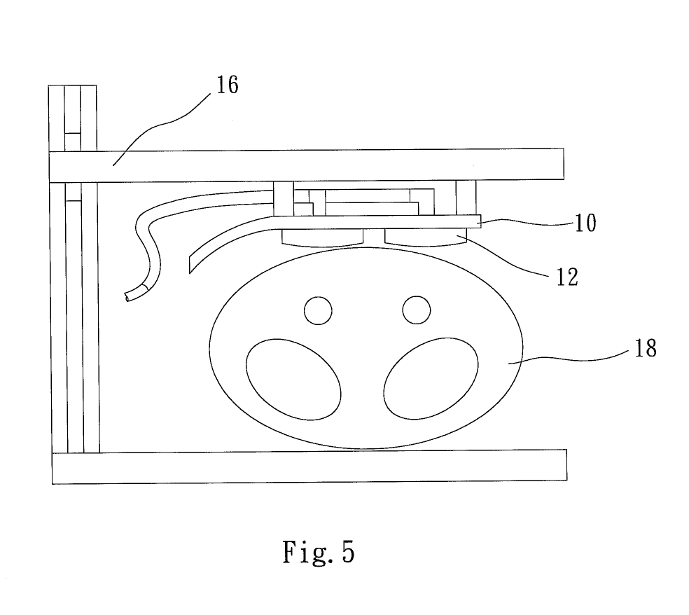

Refer to FIG. 5. When the detection device 10 is placed on the wrist 18 of a testee and the air bags 12 are inflated to have specified values of pressures, the arterial pulsation compresses the air bags 12 and causes the variation of the pressure of the air bags 12. In such a case, the pressure sensors 12a detect the pressure variations and transmit the pressure signals to the host device 20. Refer to FIG. 4A again. A pressure sensation circuit 208 of the host device 20 converts the pressure signals into electronic signals. An analog/digital converter (A/D converter) 214 receiving the electronic signals converts the electronic signals into digital signals. The microprocessor 202 connecting to the analog/digital converter 214, receives, records, and processes the digital signals. In this embodiment, the host device 20 controls the pressures of the air bags 12, measures the pressure variations of the air bags 12, and records the pulse pressure signals of the artery of the wrist 18. According the computation result, the host device 20 further controls the air pump 302 and the deflation valve 304 to operate again so as to repetitively modify the pressures of the air bags 12.

Refer to FIG. 4B. In one embodiment, the host device 20 further comprises a filter circuit 212 arranged between and electrically connected with the pressure sensation circuit 208 and the analog/digital converter 214 to increase the precision of the system. The filter circuit 212 filters out the high-frequency noise of the electronic signals output by the pressure sensation circuit 208. Thereby, the microprocessor 202 can analyze the pulse pressure signals more precisely.

Refer to FIG. 4C, which is a block diagram schematically showing the architecture of a system to measure pulse pressure signals according to another embodiment of the present invention. In this embodiment, the host device 20 further comprises a storage device 204, a communication device 206 or a display device 210, in addition to the above-mentioned microprocessor 202, analog/digital converter 214 and pressure sensation circuit 208. The storage device 204 records the measured signals. The storage device 204 may be a storage device using an SD memory card. The communication device 206 transmits the measured signals and may be a USB communication device. The display device 210 may be a graphic LCD (Liquid Crystal Display). The display device 210 presents the signals obtained by the microprocessor 202, including signals such as the realtime pulse signal, the differential signal, the integral signal, the frequency of heartbeat, or the pressures of the air bags, which are all the reference data for pulse diagnostics.

In one embodiment, the measurement system of the present invention functions to measure blood pressure. In such a case, when all the air bags 12 are enabled to be inflated, the pulse pressure signal measured by the microprocessor 202 is regarded as a blood pressure signal.

Refer to FIG. 5 again. In one embodiment, a positioning frame 16 is used to fix the relative position of the air bags 12 and the wrist 18. The positioning frame 16 may be but is not limited to a C-shape bracket. In one embodiment, the positioning frame 16 can be adjusted according to the width of the wrist 18 to make the air bags 12 compliantly contact the artery of the wrist 18. The positioning frame 16 can further secure the detection device 10 to the tested region to prevent the expanding air bags 12 from displacing the detection device 10.

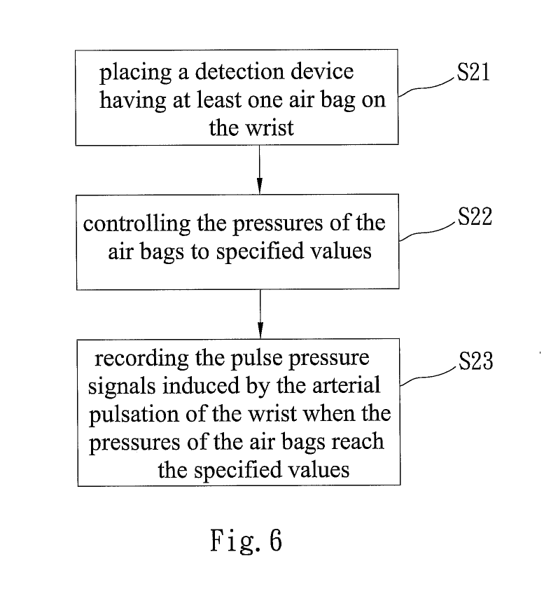

Refer to FIG. 6, which is a flowchart of a method to measure pulse pressure signals according to one embodiment of the present invention. According to the measurement system shown in FIG. 1 and FIG. 5, the measurement method of the present invention is described below. However, the measurement method using the measurement system in FIG. 1 and FIG. 5 is only an exemplification of the measurement method of the present invention. It should be understood that the measurement method of the present invention is not restricted to only apply to the measurement system in FIG. 1 and FIG. 5.

The measurement method of the present invention comprises

Step S21: placing a detection device 10 having at least one air bag 12 on the wrist 18;

Step S22: controlling the pressures of the air bags 12 to specified values; and

Step S23: the host device 20 recording the pulse pressure signals induced by the arterial pulsation of the wrist 18 when the pressures of the air bags 12 reach the specified values and the arterial pulsation of the wrist 18 compresses the air bags 12.

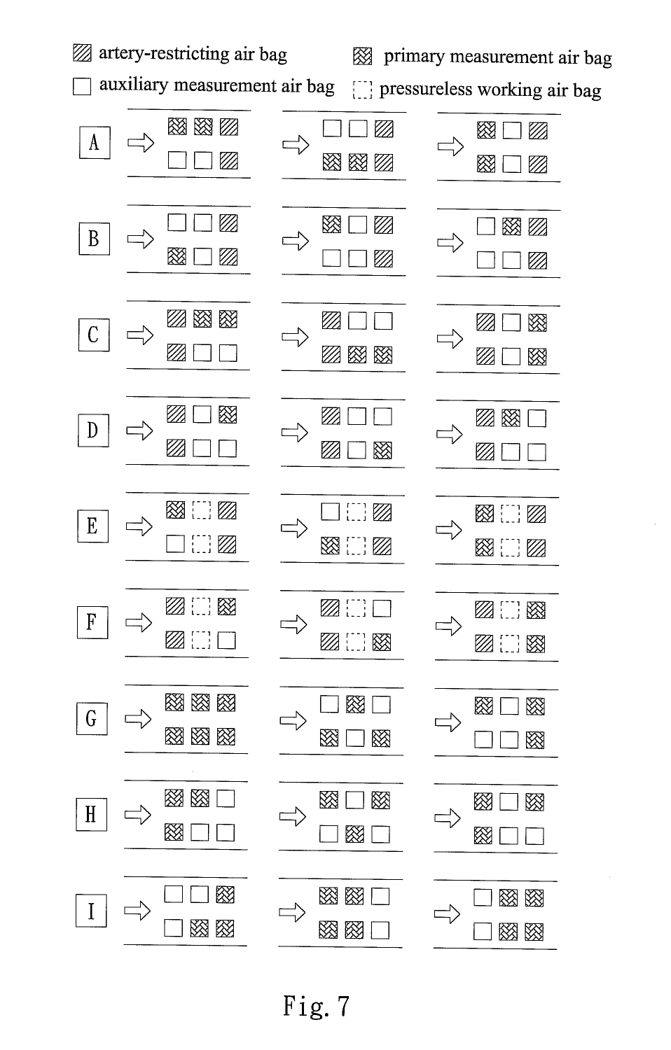

The extents of inflation of the air bag 12 are divided into four levels: none, light, medium and heavy according to specified-values pressure ranges: 0 mmHg (none), 30-80 mmHg (light), 80-120 mmHg (medium) and 120-180 mmHg (heavy). The air bags 12 of none inflation, light inflation, medium inflation and heavy inflation are respectively designated as the pressureless working air bag, auxiliary measurement air bag, primary measurement air bag, and artery-restricting air bag. The combinations of the four types of air bags 12 may be arranged to form different compression modes. FIG. 7 shows that the combinations of different types of air bags 12 are arranged into different 2×3 arrays corresponding to different compression modes respectively having different functions. For examples, Group A in FIG. 7 is to restrict the rear section of the artery and measure the pulsation signals of the front section of the artery; Group C in FIG. 7 is to restrict the front section of the artery and measure the pulsation signal of the rear section of the artery.

Furthermore, in one embodiment, when all the air bags 12 are inflated to a specified pressure, the entirety of all the air bags 12 is regarded as a complete air bag. At this time, the pulse pressure signal obtained with the complete air bag is regarded as the blood pressure signal. Therefore, the measurement system of the present invention can also be used to measure blood pressure.

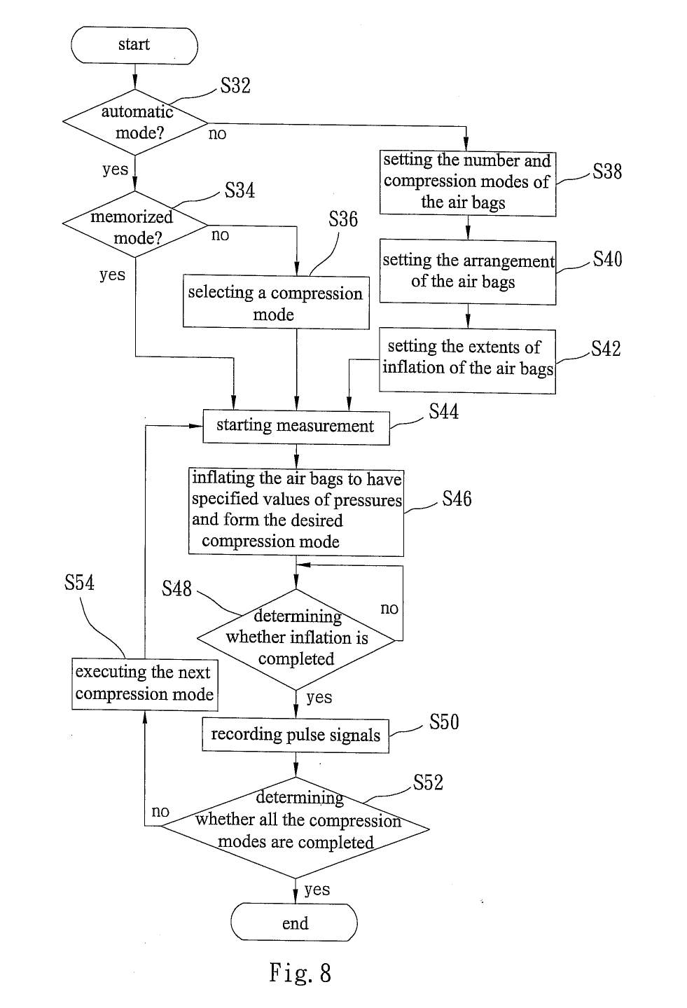

Refer to FIG. 8 for a detailed flowchart of a method to measure pulse pressure signals according to one embodiment of the present invention. The measurement method of the present invention includes an automatic mode (operated by the host device) and a manual mode (operated by an user himself). In Step 32, the system checks whether the user adopts the automatic mode. If the answer is yes, the system executes the automatic mode. In Step S34, the system checks whether the user adopts a memorized mode. If the answer is yes, the microprocessor retrieves from the memory the information of the last measurement, such as the number and compression mode of the air bags of the last measurement, and executes the same process of the last measurement. If the user adopts a non-memorized mode, the process proceeds to Step S36. In Step S36, the user, for example, selects a compression mode from FIG. 7. Then, the process proceeds to Step S44 where the measurement process starts.

If the user does not adopt the automatic mode, the process proceeds to Steps S38-S42, i.e. the system enters the manual mode. In the manual mode, the user may modify the measurement process. Therefore, the manual mode has higher flexibility than the automatic mode. For example, the user may predetermine the number and compression mode of the inflated air bags via entering a number smaller than 10 to select the pressure of each air bag and determine the extents of inflation of the air bags. Once the system confirms that the user has completed the setting of the number, pressures and compression mode of the air bags, the process proceeds to Step S44 where the measurement process starts.

In Step S46, the microprocessor controls the air pump, electromagnetic air valves and deflation valve to inflate the air bags to have specified values of pressures and form the desired compression mode. Next, in Step S48, the system checks whether the air bags have reached the specified pressures. If the answer is no, the inflation of the air bags is continued until the specified pressures are reached. If the answer is yes, the system begins to execute Step S50. In Step S50, the system measures and records the signals of human pulsation. Next, the process proceeds to Step S52. In Step S52, the system checks whether all types of measurements have completed. If the answer is no, the system continues to perform the next mode of measurement until the system confirms that all the compression modes have been completed. Then, the process ends.

The operating core of the host device can be easily realized with a single-chip microprocessor to control the system or retrieve data. Further, the system can measure signals in an automatic mode and a manual mode. In the automatic mode, the system can complete measurement in a very short time according to the preprogrammed process, wherein the compression modes corresponding to one or more combinations of the arrayed air bags can be used. In the manual mode, the user can flexibly program the system to use a new compression mode, wherein the user himself determines the number and compression mode of the arrayed air bags, whereby the system performs measurement according to the manual setting and records the measurement results in the storage device.

In conclusion, the present invention proposes a system and method to measure pulse pressure signals, which uses one or more air bags (such as an M×N array of air bags) to measure pulse signals, wherein different compression modes corresponding to different combinations and arrangements of the inflated air bags are used to restrict blood flow and measure pulse pressure. Further, the present invention can simulate the complicated finger tactics of pulse diagnostics of the traditional Chinese medicine with different combinations and arrangements of the air bags inflated to different extents.

The system and method of the present invention is not limited to measuring the pulse signals of the wrist but can also measure the pulse signals of the arteries near bones and adjacent to the skin, such as the radial artery and the dorsalis pedis artery.

The embodiments described above are to exemplify the present invention to enable the persons skilled in the art to understand, make and use the present invention. However, it is not intended to limit the scope of the present invention. Any equivalent modification or variation according to the spirit of the present invention is to be also included within the scope of the present invention.

What is claimed is:

1. A system to measure pulse signals, comprising
a detection device placed on an artery of a human limb and including at least one air bag; and
a host device controlling pressure of at least one said air bag and measuring variation of said pressure of at least one said air bag to obtain pulse signals of said artery of said human limb.

2. The system to measure pulse signals according to claim 1, wherein said detection device includes a plurality of said air bags arranged into an array.

3. The system to measure pulse signals according to claim 1 further comprising an air pump, a deflation valve and at least one electromagnetic air valve, wherein said air pump and said deflation valve are connected with at least one said electromagnetic air valve, and wherein said host device controls said air pump and said deflation valve such that at least one said electromagnetic air valve adjusts pressure of at least one said air bag.

4. The system to measure pulse signals according to claim 1, wherein said detection device includes at least one pressure sensor detecting pressure of at least one said air bag, and wherein said host device includes a pressure sensation circuit and a microprocessor, and wherein said pressure sensation circuit is connected with said pressure sensor and converting pressure signals output by said pressure sensor into electronic signals, and wherein said microprocessor records said pulse signals of said human limb according to said electronic signals.

5. The system to measure pulse signals according to claim 4, wherein said host device further includes a filter circuit, which is arranged between and electrically connected with said pressure sensation circuit and said microprocessor, and which filters out high-frequency signals from said electronic signals and implements said microprocessor to record said pulse signals of said human limb.

6. The system to measure pulse signals according to claim 4, wherein said host device further includes a storage device for storing said pulse signals recorded in said microprocessor.

7. The system to measure pulse signals according to claim 4, wherein said host device further includes a communication device for transmitting said pulse signals recorded by said microprocessor.

8. The system to measure pulse signals according to claim 1, wherein each said air bag includes an inlet, a top cover, a bottom cover, and a plurality of folding laminate members, and wherein said host device adjusts pressure of each said air bag via said inlet, and wherein said folding laminate member is arranged between said top cover and said bottom cover to increase volume of each said air bag having been expanded.

9. The system to measure pulse signals according to claim 1 further comprising a positioning frame used to fix said detection device and secure a relative position of at least one said air bag and said human limb.

10. The system to measure pulse signals according to claim 1, wherein when all said air bags are enabled to be inflated, said pulse signals of said human limb are regarded as blood pressure signals.

11. A method to measure pulse signals, comprising steps:
placing a detection device having at least one air bag on an artery of a human limb;
controlling at least one said air bag to have a specified value of pressure; and
recording pulse signals of said artery of said human limb when pressure of at least one said air bag has reached said specified value.

12. The method to measure pulse signals according to claim 11 further comprising a step of determining a count and inflation extent of at least one said air bag before controlling at least one said air bag to have said specified value of pressure.

13. The method to measure pulse signals according to claim 11 further comprising a step of retrieving a count and inflation extent of at least one said air bag inflated last time and inflating at least one said air bag according to said count and said inflation extent before controlling at least one said air bag to have said specified value of pressure.

14. The method to measure pulse signals according to claim 11, wherein at least one said air bag is controlled to have a specified value of pressure manually by a user or automatically by a host device.

15. The method to measure pulse signals according to claim 11, wherein when pressure of one said air bag does not reach said specified value, said air bag is inflated continuously.

16. The method to measure pulse signals according to claim 11, wherein when all said air bags are inflated to a specified value of pressure, entirety of said air bags is regarded as a complete air bag able to measure blood pressure.
